(19) United States
(12) Patent Application Publication
(10) Pub. No.: US 2003/0092039 A1
(43) Pub. Date: May 15, 2003

(54) Screening nutraceuticals

(75) Inventors: Marilyn C. Olson-Munoz, Madison, WI (US); Glen Donald, Madison, WI (US)

Correspondence Address: Jason R. Bond, MEDLEN & CARROLL, LLP, Suite 350, 101 Howard Street, San Francisco, CA, 94105, US

(73) Assignee: Third Wave Technologies, Inc., Madison, WI

(21) Appl. No.: 10/210,682

(22) Filed: Aug. 1, 2002

Related U.S. Application Data
(60) Provisional application No. 60/309,279, filed on Aug. 1, 2001.

(51) Int. Cl. C12Q 1/68; A61K 35/78

(52) U.S. Cl. 435/6; 424/725

(57) ABSTRACT

The present invention relates to screening assays for testing nutraceuticals. In particular, the present invention relations to methods for detecting RNA is cells exposed to various types, concentrations, and combinations of nutraceuticals employing the INVADER detection assay. In preferred embodiments, the screening assays of the present invention are configured for high throughput screening.

The present Application claims priority to U.S. Provisional Application Serial No. 60/309,279, filed Aug. 1, 2001, herein incorporated by reference.

### FIELD OF THE INVENTION

The present invention relates to screening assays for testing nutraceuticals. In particular, the present invention relations to methods for detecting RNA is cells exposed to various types, concentrations, and combinations of nutraceuticals employing the INVADER detection assay. In preferred embodiments, the screening assays of the present invention are configured for high throughput screening.

### BACKGROUND OF THE INVENTION

In its broadest definition, the U.S. nutraceutical market today is estimated at $250 billion, 50% of the U.S. food market, if one includes dietary supplements, vitamin fortified products, sugar substitutes, fat substitutes, fiber-enriched foods, vegetables, fatless meats, skim milk, low-calorie diets, etc. The market is growing rapidly as affluent baby boomers grow older, conventional healthcare costs escalate, consumers are becoming more aware of the health benefits of certain foods and are becoming more concerned with the delivery of conventional health services (The Nutraceuticals Institute, Nutraceuticals/Functional Foods/Biotech 2000).

The nutraceutical industry is still in its early stages, but is still attracting the interest of large pharmaceutical and food companies because of its size and growth potential in the health and wellness market. These organizations offer strong science, strong market presence, and strong brand equity. In order for products to reach their full commercial potential as nutraceuticals, in the face of increased consumer education on heath issues, they will require that health claims be supported by legitimate scientific evidence demonstrating the bioactivity of the molecules or substances being marketed. What is needed, therefore, is a high throughput, sensitive, quantitative and cost-effective platform for measuring gene expression in response to natural compounds (e.g. derived from food), such a nutraceuticals.

### SUMMARY OF THE INVENTION

The present invention relates to screening assays for testing nutraceuticals. In particular, the present invention relations to methods for detecting RNA is cells exposed to various types, concentrations, and combinations of nutraceuticals employing the INVADER detection assay. In preferred embodiments, the screening assays of the present invention are configured for high throughput screening.

In some embodiments, the present invention provides methods for testing a nutraceutical, comprising; a) providing; i) cells, wherein the cells express a baseline level of test gene mRNA, ii) a nutraceutical; and iii) INVADER assay detection reagents configured for detecting and quantitating the test gene mRNA; and b) exposing the cells to the nutraceutical, c) lysing the cells such that a cell lysate is generated, and d) contacting the cell lysate with the INVADER assay detection reagents under conditions such that an assayed level of the test gene mRNA is determined. In other embodiments, the method further comprises step e) comparing the baseline level of the test gene mRNA to the assayed level of the test gene mRNA.

In particular embodiments, the methods for testing a nutraceuticals, comprise; a) providing; i) cells, wherein the cells express a baseline level of test gene mRNA, and wherein the cells express a first level of an internal reference gene mRNA, ii) a nutraceutical; iii) INVADER assay detection reagents configured for detecting and quantitating the test gene mRNA and the internal reference gene mRNA; and b) exposing the cells to the nutraceutical, and c) lysing the cells such that a cell lysate is generated, and d) contacting the cell lysate with the INVADER assay detection reagents under conditions such that an assayed level of the test gene mRNA is determined, and such that a second level of the internal reference gene is determined. In some embodiments, the method further comprises step e) comparing the first level of the internal reference gene to the second level of the internal reference gene, and comparing the baseline level of the test gene mRNA to the assayed level of the test gene mRNA.

In certain embodiments, the present invention provides methods for testing a nutraceutical, comprising; a) providing; i) a population of cells expressing test gene mRNA, ii) a nutraceutical; iii) INVADER assay detection reagents configured for detecting and quantitating the test gene mRNA; and b) lysing a first portion of the population of cells such that a first cell lysate is generated, c) contacting the first cell lysate with the INVADER assay detection reagents under conditions such that a baseline level of the test gene mRNA is determined, d) exposing a second portion of the population of cells to the nutraceutical, e) lysing the second portion of the population of cells such that a second cell lysate is generated, and f) contacting the second cell lysate with the INVADER assay detection reagents under conditions such that an assayed level of the test gene mRNA is determined.

In particular embodiments, the comparing generates nutraceutical activity data for the nutraceutical (e.g. data concerning the biochemical effect of the nutraceutical on the type of cell tested, which may be relevant to treating or preventing certain human diseases). In preferred embodiments, the method further comprises step of employing the nutraceutical activity data to substantiate a structure/function claim (as the term is used in the Dietary Supplement Health and Enforcement Act of 1994). For example, the nutraceutical activity data may be submitted to the Food and Drug Administration to support a structure/functional claim such that the product can be properly labeled and sold in the United States.

In certain embodiments, the test gene mRNA comprises nitric oxide synthase mRNA. In other embodiments, the test gene mRNA comprises any type of mRNA who's expression may be modulated by a nutruaceutical (including splice variants of particular genes). In preferred embodiments, the nitric oxide synthase mRNA is human (iNOS/NOS2A, see SEQ ID NO:1 in FIG. 2). In some embodiments, the INVADER assay detection reagents comprise a probe and an INVADER oligonucleotide, and may further comprise FRET cassettes, a structure specific enzyme, etc. In preferred embodiments, the INVADER assay detection reagents are optimized for detecting and quantitating both test gene mRNA and internal reference mRNA (biplex), such that the reaction can be carried out in a single well). In certain embodiments, the nutraceutical is classified as a Dietary Supplement under the Dietary Supplement Health and Education Act (DSHEA) of 1994. In some embodiments, the lysing comprises heating the cells to a temperature of approximately 80-90 degrees Celsius. In preferred embodiments, the contacting is performed in a high throughput manner. In other embodiments, the exposing, the lysing, and the contacting are performed in an automated manner (e.g. with robotic equipment).

In other embodiments, the present invention provides methods for testing a nutraceutical, comprising; a) providing; i) a surface comprising a plurality of spatially discrete regions, wherein the spatially discrete regions comprise cells, wherein the cells express a baseline level of test gene mRNA, ii) at least one type of nutraceutical; and iii) INVADER assay detection reagents configured for detecting and quantitating the test gene mRNA; and b) adding the at least one type of nutraceutical to at least two of the plurality of spatially discrete regions, c) lysing the cells in the at least two of the plurality of spatially discrete regions, and d) contacting the at least two of the plurality of spatially discrete regions with the INVADER assay detection reagents under conditions such that an assayed level of the test gene mRNA is determined for the cells in each of the at least two of the plurality of spatially discrete regions. In some embodiments, the plurality of spatially discrete regions are wells (e.g. in a 96-well plate).

### DESCRIPTION OF THE FIGURES

FIG. 1 shows a schematic of the INVADER assay. FIG. 1A shows the standard INVADER assay reaction (top), as well as the INVADER assay ‘squared’ format with the secondary FRET cassette (bottom).

FIG. 2 shows an mRNA sequence for human nitric oxide synthase 2A (SEQ ID NO:1). The corresponding cDNA is accession no. NM_{—}000626 (See, Geller et al, Proc. Natl. Acad. Sci., USA, 90 (8), 3491-3495 (1993, hereby incorporated by reference).

FIG. 3 shows a graph of the production of aP2 mRNA in cells treated with seven different compounds at six different concentrations.

### DEFINITIONS

To facilitate an understanding of the invention, a number of terms are defined below.

As used herein, the term “nutraceutical” is used to refer to any substance that is a food, part of a food, or a ‘dietary supplement’ (as used under the Dietary Supplemental Health and Education Act), and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingrediants, and a stimulant functional food or pharmafood.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand. Nucleotide analogs used to form non-standard base pairs, whether with another nucleotide analog (e.g., an IsoC/IsoG base pair), or with a naturally occurring nucleotide (e.g., as described in U.S. Pat. No. 5,912,340, herein incorporated by reference in its entirety) are also considered to be complementary to a base pairing partner within the meaning this definition.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

With regard to complementarity, it is important for some diagnostic applications to determine whether the hybridization represents complete or partial complementarity. For example, where it is desired to detect simply the presence or absence of pathogen DNA (such as from a virus, bacterium, fungi, mycoplasma, protozoan) it is only important that the hybridization method ensures hybridization when the relevant sequence is present; conditions can be selected where both partially complementary probes and completely complementary probes will hybridize. Other diagnostic applications, however, may require that the hybridization method distinguish between partial and complete complementarity. It may be of interest to detect genetic polymorphisms. For example, human hemoglobin is composed, in part, of four polypeptide chains. Two of these chains are identical chains of 141 amino acids (alpha chains) and two of these chains are identical chains of 146 amino acids (beta chains). The gene encoding the beta chain is known to exhibit polymorphism. The normal allele encodes a beta chain having glutamic acid at the sixth position. The mutant allele encodes a beta chain having valine at the sixth position. This difference in amino acids has a profound (most profound when the individual is homozygous for the mutant allele) physiological impact known clinically as sickle cell anemia. It is well known that the genetic basis of the amino acid change involves a single base difference between the normal allele DNA sequence and the mutant allele DNA sequence.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G. T mismatches in DNA. Biochemistry 36, 10581-94 (1997) include more sophisticated computations which take structural and environmental, as well as sequence characteristics into account for the calculation of Tₘ.

As used herein the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another be hybridized or annealed together.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full-length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be the to have 5′ and 3′ ends. A first region along a nucleic acid strand is the to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc.) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection systems known in the art, or combinations thereof.

The term “cleavage structure” as used herein, refers to a structure that is formed by the interaction of at least one probe oligonucleotide and a target nucleic acid, forming a structure comprising a duplex, the resulting structure being cleavable by a cleavage agent, including but not limited to an enzyme. The cleavage structure is a substrate for specific cleavage by the cleavage means in contrast to a nucleic acid molecule that is a substrate for non-specific cleavage by agents such as phosphodiesterases that cleave nucleic acid molecules without regard to secondary structure (i.e., no formation of a duplexed structure is required).

The term “folded cleavage structure” as used herein, refers to a region of a single-stranded nucleic acid substrate containing secondary structure, the region being cleavable by an enzymatic cleavage means. The cleavage structure is a substrate for specific cleavage by the cleavage means in contrast to a nucleic acid molecule that is a substrate for non-specific cleavage by agents such as phosphodiesterases that cleave nucleic acid molecules without regard to secondary structure (i.e., no folding of the substrate is required).

As used herein, the term “folded target” refers to a nucleic acid strand that contains at least one region of secondary structure (i.e., at least one double stranded region and at least one single-stranded region within a single strand of the nucleic acid). A folded target may comprise regions of tertiary structure in addition to regions of secondary structure.

The term “cleavage means” or “cleavage agent” as used herein refers to any means that is capable of cleaving a cleavage structure, including but not limited to enzymes. The cleavage means may include native DNAPs having 5′ nuclease activity (e.g., Taq DNA polymerase, E. coli DNA polymerase I) and, more specifically, modified DNAPs having 5′ nuclease but lacking synthetic activity. “Structure-specific nucleases” or “structure-specific enzymes” are enzymes that recognize specific secondary structures in a nucleic acid molecule and cleave these structures. The cleavage means of the invention cleave a nucleic acid molecule in response to the formation of cleavage structures; it is not necessary that the cleavage means cleave the cleavage structure at any particular location within the cleavage structure.

The cleavage means is not restricted to enzymes having solely 5′ nuclease activity. The cleavage means may include nuclease activity provided from a variety of sources including the CLEAVASE enzymes, the FEN-1 endonucleases (including RAD2 and XPG proteins), Taq DNA polymerase and E. coli DNA polymerase I.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage means with a cleavage structure (i.e., the treatment of a cleavage structure with a cleavage means).

The term “target nucleic acid” refers to a nucleic acid molecule containing a sequence that has at least partial complementarity with at least a probe oligonucleotide and may also have at least partial complementarity with an INVADER oligonucleotide. The target nucleic acid may comprise single- or double-stranded DNA or RNA, and may comprise nucleotide analogs, labels, and other modifications. In preferred embodiments, the target is mRNA.

The term “probe oligonucleotide” refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “non-target cleavage product” refers to a product of a cleavage reaction that is not derived from the target nucleic acid. As discussed above, in the methods of the present invention, cleavage of the cleavage structure generally occurs within the probe oligonucleotide. The fragments of the probe oligonucleotide generated by this target nucleic acid-dependent cleavage are “non-target cleavage products.”

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide—whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “substantially single-stranded” when used in reference to a nucleic acid substrate means that the substrate molecule exists primarily as a single strand of nucleic acid in contrast to a double-stranded substrate which exists as two strands of nucleic acid which are held together by inter-strand base pairing interactions.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are the to vary in sequence from one another. A second mutant form of the structural gene may exist. This second mutant form is the to vary in sequence from both the wild-type gene and the first mutant form of the gene.

The term “liberating” as used herein refers to the release of a nucleic acid fragment from a larger nucleic acid fragment, such as an oligonucleotide, by the action of, for example, a 5′ nuclease such that the released fragment is no longer covalently attached to the remainder of the oligonucleotide.

The term “Kₘ” as used herein refers to the Michaelis-Menten constant for an enzyme and is defined as the concentration of the specific substrate at which a given enzyme yields one-half its maximum velocity in an enzyme catalyzed reaction.

The term “nucleotide” as used herein includes, but is not limited to, naturally occurring and/or synthetic nucleotides, nucleotide analogs, and nucleotide derivatives. For example, the term includes naturally occurring DNA or RNA monomers, nucleotides with backbone modifications such as peptide nucleic acid (PNA) (M. Egholm et al., Nature 365:566 [1993]), phosphorothioate DNA, phosphorodithioate DNA, phosphoramidate DNA, aminde-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-chloro-, iodo-, methyl-, ethyl-, vinyl-, formyl-, ethynyl-, propynyl-, alkynyl-, thiazoyl-, imidazolyl-, pyridyl-), 7-deazapurines with C-7 substituents including fluoro-, bromo-, chloro-, iodo-, methyl-, ethyl-, vinyl-, formyl-, alkynyl-, alkenyl-, thiazolyl-, imidazolyl-, pyridyl-), inosine and diaminopurine.

The term “base analog” as used herein refers to modified or non-naturally occurring bases such as 7-deaza purines (e.g., 7-deaza-adenine and 7-deaza-guanine); bases modified, for example, to provide altered interactions such as non-standard basepairing, including, but not limited to: IsoC, Iso G, and other modified bases and nucleotides described in U.S. Pat. Nos. 5,432,272; 6,001,983; 6,037,120; 6,140,496; 5,912,340; 6,127,121 and 6,143,877, each of which is incorporated herein by reference in their entireties; heterocyclic base analogs based no the purine or pyrimidine ring systems, and other heterocyclic bases. Nucleotide analogs include base analogs and comprise modified forms of deoxyribonucleotides as well as ribonucleotides.

The term “polymorphic locus” is a locus present in a population that shows variation between members of the population (e.g., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The term “source of target nucleic acid” refers to any sample that contains nucleic acids (RNA or DNA). Particularly preferred sources of target nucleic acids are biological samples including, but not limited to tissue cultures, blood, saliva, cerebral spinal fluid, pleural fluid, milk, lymph, sputum and semen.

An oligonucleotide is the to be present in “excess” relative to another oligonucleotide (or target nucleic acid sequence) if that oligonucleotide is present at a higher molar concentration that the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present at least a 100-fold molar excess; typically at least 1 pmole of each probe oligonucleotide would be used when the target nucleic acid sequence was present at about 10 fmoles or less.

A sample “suspected of containing” a first and a second target nucleic acid may contain either, both or neither target nucleic acid molecule.

The term “charge-balanced” oligonucleotide refers to an oligonucleotide (the input oligonucleotide in a reaction) that has been modified such that the modified oligonucleotide bears a charge, such that when the modified oligonucleotide is either cleaved (i.e., shortened) or elongated, a resulting product bears a charge different from the input oligonucleotide (the “charge-unbalanced” oligonucleotide) thereby permitting separation of the input and reacted oligonucleotides on the basis of charge. The term “charge-balanced” does not imply that the modified or balanced oligonucleotide has a net neutral charge (although this can be the case). Charge-balancing refers to the design and modification of an oligonucleotide such that a specific reaction product generated from this input oligonucleotide can be separated on the basis of charge from the input oligonucleotide.

For example, in an INVADER oligonucleotide-directed cleavage assay in which the probe oligonucleotide bears the sequence: 5′ TTCTTTTCACCAGCGAGACGGG 3′ (i.e., SEQ ID NO:136 without the modified bases) and cleavage of the probe occurs between the second and third residues, one possible charge-balanced version of this oligonucleotide would be: 5′ Cy3-AminoT-Amino-TCTTTTCACCAGCGAGAC GGG 3′. This modified oligonucleotide bears a net negative charge. After cleavage, the following oligonucleotides are generated: 5′ Cy3-AminoT-Amino-T 3′ and 5′ CTTTTCACCAGCGAGACGGG 3′ (residues 3-22 of SEQ ID NO:136). 5′ Cy3-AminoT-Amino-T 3′ bears a detectable moiety (the positively-charged Cy3 dye) and two amino-modified bases. The amino-modified bases and the Cy3 dye contribute positive charges in excess of the negative charges contributed by the phosphate groups and thus the 5′ Cy3-AminoT-Amino-T 3′oligonucleotide has a net positive charge. The other, longer cleavage fragment, like the input probe, bears a net negative charge. Because the 5′ Cy3-AminoT-Amino-T 3′fragment is separable on the basis of charge from the input probe (the charge-balanced oligonucleotide), it is referred to as a charge-unbalanced oligonucleotide. The longer cleavage product cannot be separated on the basis of charge from the input oligonucleotide as both oligonucleotides bear a net negative charge; thus, the longer cleavage product is not a charge-unbalanced oligonucleotide.

The term “net neutral charge” when used in reference to an oligonucleotide, including modified oligonucleotides, indicates that the sum of the charges present (i.e., R—NH3+ groups on thymidines, the N3 nitrogen of cytosine, presence or absence or phosphate groups, etc.) under the desired reaction or separation conditions is essentially zero. An oligonucleotide having a net neutral charge would not migrate in an electrical field.

The term “net positive charge” when used in reference to an oligonucleotide, including modified oligonucleotides, indicates that the sum of the charges present (i.e., R—NH3+ groups on thymidines, the N3 nitrogen of cytosine, presence or absence or phosphate groups, etc.) under the desired reaction conditions is +1 or greater. An oligonucleotide having a net positive charge would migrate toward the negative electrode in an electrical field.

The term “net negative charge” when used in reference to an oligonucleotide, including modified oligonucleotides, indicates that the sum of the charges present (i.e., R—NH3+ groups on thymidines, the N3 nitrogen of cytosine, presence or absence or phosphate groups, etc.) under the desired reaction conditions is −1 or lower. An oligonucleotide having a net negative charge would migrate toward the positive electrode in an electrical field.

The term “polymerization means” or “polymerization agent” refers to any agent capable of facilitating the addition of nucleoside triphosphates to an oligonucleotide. Preferred polymerization means comprise DNA and RNA polymerases.

The term “ligation means” or “ligation agent” refers to any agent capable of facilitating the ligation (i.e., the formation of a phosphodiester bond between a 3′-OH and a 5′ P located at the termini of two strands of nucleic acid). Preferred ligation means comprise DNA ligases and RNA ligases.

The term “reactant” is used herein in its broadest sense. The reactant can comprise, for example, an enzymatic reactant, a chemical reactant or light (e.g., ultraviolet light, particularly short wavelength ultraviolet light is known to break oligonucleotide chains). Any agent capable of reacting with an oligonucleotide to either shorten (i.e., cleave) or elongate the oligonucleotide is encompassed within the term “reactant.”

The term “adduct” is used herein in its broadest sense to indicate any compound or element that can be added to an oligonucleotide. An adduct may be charged (positively or negatively) or may be charge-neutral. An adduct may be added to the oligonucleotide via covalent or non-covalent linkages. Examples of adducts include, but are not limited to, indodicarbocyanine dye amidites, amino-substituted nucleotides, ethidium bromide, ethidium homodimer, (1,3-propanediamino)propidium, (diethylenetriamino)propidium, thiazole orange, (N-N′-tetramethyl-1,3-propanediamino)propyl thiazole orange, (N-N′-tetramethyl-1,2-ethanediamino)propyl thiazole orange, thiazole orange-thiazole orange homodimer (TOTO), thiazole orange-thiazole blue heterodimer (TOTAB), thiazole orange-ethidium heterodimer 1 (TOED1), thiazole orange-ethidium heterodimer 2 (TOED2) and fluorescein-ethidium heterodimer (FED), psoralens, biotin, streptavidin, avidin, etc.

Where a first oligonucleotide is complementary to a region of a target nucleic acid and a second oligonucleotide has complementary to the same region (or a portion of this region) a “region of sequence overlap” exists along the target nucleic acid. The degree of overlap will vary depending upon the nature of the complementarity (see, e.g., region “X” in FIGS. 29 and 67 and the accompanying discussions).

As used herein, the term “purified” or “to purify” refers to the removal of contaminants from a sample. For example, recombinant CLEAVASE nucleases are expressed in bacterial host cells and the nucleases are purified by the removal of host cell proteins; the percent of these recombinant nucleases is thereby increased in the sample.

The term “recombinant DNA molecule” as used herein refers to a DNA molecule that comprises of segments of DNA joined together by means of molecular biological techniques.

The term “recombinant protein” or “recombinant polypeptide” as used herein refers to a protein molecule that is expressed from a recombinant DNA molecule.

As used herein the term “portion” when in reference to a protein (as in “a portion of a given protein”) refers to fragments of that protein. The fragments may range in size from four amino acid residues to the entire amino acid sequence minus one amino acid (e.g., 4, 5, 6, . . ., n-1).

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

The term “continuous strand of nucleic acid” as used herein is means a strand of nucleic acid that has a continuous, covalently linked, backbone structure, without nicks or other disruptions. The disposition of the base portion of each nucleotide, whether base-paired, single-stranded or mismatched, is not an element in the definition of a continuous strand. The backbone of the continuous strand is not limited to the ribose-phosphate or deoxyribose-phosphate compositions that are found in naturally occurring, unmodified nucleic acids. A nucleic acid of the present invention may comprise modifications in the structure of the backbone, including but not limited to phosphorothioate residues, phosphonate residues, 2′ substituted ribose residues (e.g., 2′-O-methyl ribose) and alternative sugar (e.g., arabinose) containing residues.

The term “continuous duplex” as used herein refers to a region of double stranded nucleic acid in which there is no disruption in the progression of basepairs within the duplex (i.e., the base pairs along the duplex are not distorted to accommodate a gap, bulge or mismatch with the confines of the region of continuous duplex). As used herein the term refers only to the arrangement of the basepairs within the duplex, without implication of continuity in the backbone portion of the nucleic acid strand. Duplex nucleic acids with uninterrupted basepairing, but with nicks in one or both strands are within the definition of a continuous duplex.

The term “duplex” refers to the state of nucleic acids in which the base portions of the nucleotides on one strand are bound through hydrogen bonding the their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

The term “duplex dependent protein binding” refers to the binding of proteins to nucleic acid that is dependent on the nucleic acid being in a duplex, or helical form.

The term “duplex dependent protein binding sites or regions” as used herein refers to discrete regions or sequences within a nucleic acid that are bound with particular affinity by specific duplex-dependent nucleic acid binding proteins. This is in contrast to the generalized duplex-dependent binding of proteins that are not site-specific, such as the histone proteins that bind chromatin with little reference to specific sequences or sites.

The term “protein-binding region” as used herein refers to a nucleic acid region identified by a sequence or structure as binding to a particular protein or class of proteins. It is within the scope of this definition to include those regions that contain sufficient genetic information to allow identifications of the region by comparison to known sequences, but which might not have the requisite structure for actual binding (e.g., a single strand of a duplex-depending nucleic acid binding protein site). As used herein “protein binding region” excludes restriction endonuclease binding regions.

The term “complete double stranded protein binding region” as used herein refers to the minimum region of continuous duplex required to allow binding or other activity of a duplex-dependent protein. This definition is intended to encompass the observation that some duplex dependent nucleic acid binding proteins can interact with full activity with regions of duplex that may be shorter than a canonical protein binding region as observed in one or the other of the two single strands. In other words, one or more nucleotides in the region may be allowed to remain unpaired without suppressing binding. As used here in, the term “complete double stranded binding region” refers to the minimum sequence that will accommodate the binding function. Because some such regions can tolerate non-duplex sequences in multiple places, although not necessarily simultaneously, a single protein binding region might have several shorter sub-regions that, when duplexed, will be fully competent for protein binding.

The term “template” refers to a strand of nucleic acid on which a complementary copy is built from nucleoside triphosphates through the activity of a template-dependent nucleic acid polymerase. Within a duplex the template strand is, by convention, depicted and described as the “bottom” strand. Similarly, the non-template strand is often depicted and described as the “top” strand.

The term “template-dependent RNA polymerase” refers to a nucleic acid polymerase that creates new RNA strands through the copying of a template strand as described above and which does not synthesize RNA in the absence of a template. This is in contrast to the activity of the template-independent nucleic acid polymerases that synthesize or extend nucleic acids without reference to a template, such as terminal deoxynucleotidyl transferase, or Poly A polymerase.

The term “ARRESTOR molecule” refers to an agent added to or included in an invasive cleavage reaction in order to stop one or more reaction components from participating in a subsequent action or reaction. This may be done by sequestering or inactivating some reaction component (e.g., by binding or base-pairing a nucleic acid component, or by binding to a protein component). The term “ARRESTOR oligonucleotide” refers to an oligonucleotide included in an invasive cleavage reaction in order to stop or arrest one or more aspects of any reaction (e.g., the first reaction and/or any subsequent reactions or actions; it is not intended that the ARRESTOR oligonucleotide be limited to any particular reaction or reaction step). This may be done by sequestering some reaction component (e.g., base-pairing to another nucleic acid, or binding to a protein component). However, it is not intended that the term be so limited as to just situations in which a reaction component is sequestered.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “functional domain” refers to a region, or a part of a region, of a protein (e.g., an enzyme) that provides one or more functional characteristic of the protein. For example, a functional domain of an enzyme may provide, directly or indirectly, one or more activities of the enzyme including, but not limited to, substrate binding capability and catalytic activity. A functional domain may be characterized through mutation of one or more amino acids within the functional domain, wherein mutation of the amino acid(s) alters the associated functionality (as measured empirically in an assay) thereby indicating the presence of a functional domain.

As used herein, the term “heterologous functional domain” refers to a protein functional domain that is not in its natural environment. For example, a heterologous functional domain includes a functional domain from one enzyme introduced into another enzyme. A heterologous functional domain also includes a functional domain native to a protein that has been altered in some way (e.g., mutated, added in multiple copies, etc.). A heterologous functional domain may comprise a plurality of contiguous amino acids or may include two or more distal amino acids are amino acids fragments (e.g., two or more amino acids or fragments with intervening, non-heterologous, sequence). Heterologous functional domains are distinguished from endogenous functional domains in that the heterologous amino acid(s) are joined to or contain amino acid sequences that are not found naturally associated with the amino acid sequence in nature or are associated with a portion of a protein not found in nature.

### DESCRIPTION OF THE INVENTION

Quantitation of specific RNAs has emerged as an accurate, predictive indicator of numerous clinical conditions and therapeutic efficacy. Because mRNA expression precedes protein synthesis, the advent of a scaleable technology that directly quantitates specific mRNAs can accelerate high-throughput nutraceutical screening beyond reliance on protein expression. The ability to screen for therapeutic effects using gene expression rather than protein accumulation also reduces artifacts that arise when cells are incubated in the presence of high concentrations of test compounds for long periods. Despite this need, most products focus on assessing global differences in gene expression and relatively few techniques for measuring specific mRNA levels have emerged that are truly direct, quantitative, and amenable to high-throughput analysis.

The INVADER assay is well suited for detection of mRNA in a high throughput system. Although previous methods can be sensitive, they fail to quantitate and distinguish closely related mRNAs accurately, especially those expressed at different levels in the same sample. The INVADER assay provides many advantages, making it highly useful for screening of mRNA. These advantages include the following:

Cost effectiveness—material costs for this assay are 10 to 20 fold less than for existing gene expression tests.

Ease of use—The assay has two simple steps and is homogenous.

Compatible with automation—The formats generally used for this assay are well suited for high throughput screening.

Adaptability and flexibility—The INVADER assay generally used common laboratory reagents and supplies and can be configured for microtiter plate-based readout on a variety of low-cost instrument platforms.

Large dynamic range—the linear range of detection for this assay is at least three orders of magnitude. Using both continuous and discrete time point kinetic readouts, the range may be extended (e.g. to seven orders of magnitude).

Sensitivity—This assay can detect as little as, for example, 0.003 amoles (1800 molecules) RNA and detect changes in RNA levels as small as, for example, 1.2 fold.

Precision—intra-assay CVs are typically<5%, permitting accurate quantitation of small changes in expression levels.

Discrimination—This assay can discriminate among single base differences and is ideal for distinguishing closely related mRNAs.

I. INVADER Assays

The INVADER assay detects hybridization of probes to a target by enzymatic cleavage of specific structures by structure specific enzymes (See, INVADER assays, Third Wave Technologies; See e.g., U.S. Pat. Nos. 5,846,717; 6,090,543; 6,001,567; 5,985,557; 5,994,069; Lyamichev et al., Nat. Biotech., 17:292 (1999), Hall et al., PNAS, USA, 97:8272 (2000), WO97/27214 and WO9842873, each of which is herein incorporated by reference in their entirety for all purposes). INVADER detection assays, INVADER assay components (e.g. probe, INVADER oligonucleotide, and structure specific cleavage enzymes), as well as services for designing particular INVADER assays, are available commercially from Third Wave Technologies, Madison, Wis.

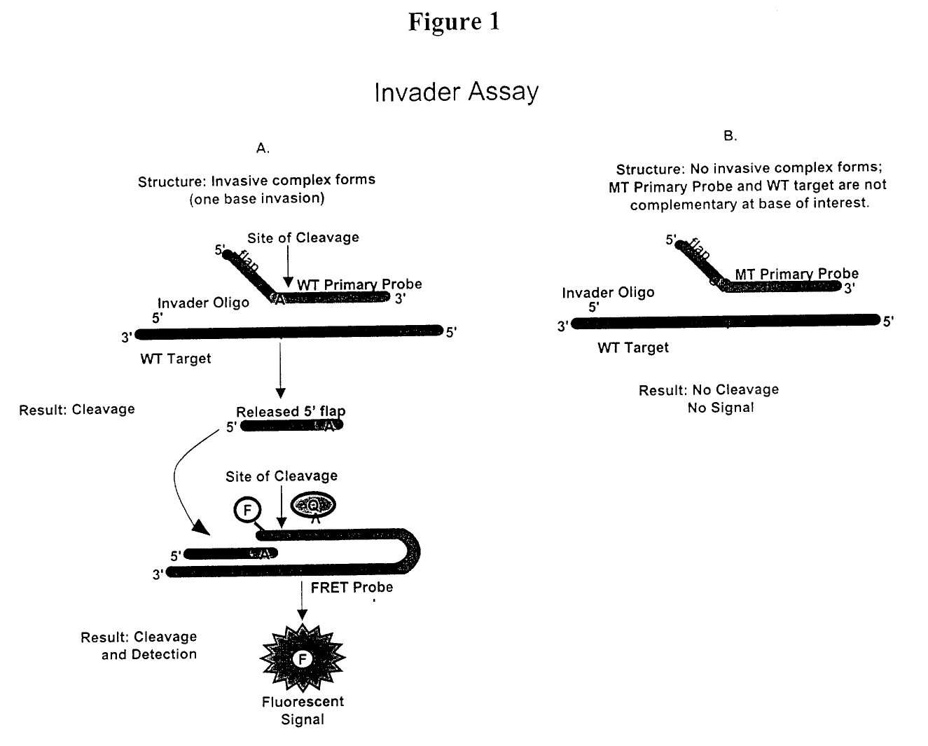

The INVADER assay detects specific DNA and RNA sequences by using structure-specific enzymes (e.g. FEN endonucleases) to cleave a complex formed by the hybridization of overlapping oligonucleotide probes (See, e.g. FIG. 1). Elevated temperature and an excess of one of the probes enable multiple probes to be cleaved for each target sequence present without temperature cycling. In some embodiments, these cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified, as well as amplified, genomic DNA. In the embodiments shown schematically in FIG. 1, the INVADER assay uses two cascading steps (a primary and a secondary reaction) both to generate and then to amplify the target-specific signal. For convenience, the alleles in the following discussion are described as wild-type (WT) and mutant (MT), even though this terminology does not apply to all genetic variations. In the primary reaction (FIG. 1, top of panel A), the WT primary probe and the INVADER oligonucleotide hybridize in tandem to the target nucleic acid to form an overlapping structure. An unpaired “flap” is included on the 5′ end of the WT primary probe. A structure specific enzyme (e.g. the CLEAVASE enzyme, Third Wave Technologies) recognizes the overlap and cleaves off the unpaired flap, releasing it as a target-specific product. In the secondary reaction (INVADER squared reaction), this cleaved product serves as an INVADER oligonucleotide on the WT fluorescence resonance energy transfer (WT-FRET) probe to again create the structure recognized by the structure specific enzyme (panel A). When the two dyes on a single FRET probe are separated by cleavage (indicated by the arrow in FIG. 1), a detectable fluorescent signal above background fluorescence is produced. Consequently, cleavage of this second structure results in an increase in fluorescence, indicating the presence of the WT allele (or mutant allele if the assay is configured for the mutant allele to generate the detectable signal). If the primary probe oligonucleotide and the target nucleotide sequence do not match perfectly at the cleavage site (e.g., as with the MT primary probe and the WT target, FIG. 1, panel B), the overlapped structure does not form and cleavage is suppressed. The structure specific enzyme (e.g. CLEAVASE VIII enzyme, Third Wave Technologies) used cleaves the overlapped structure more efficiently (e.g. at least 340-fold) than the non-overlapping structure, allowing excellent discrimination of the alleles. The probes turn over without temperature cycling to produce many signals per target (i.e., linear signal amplification). Similarly, each target-specific product can enable the cleavage of many FRET probes.

The primary INVADER assay reaction is directed against the target RNA (or DNA) being detected. The target RNA is the limiting component in the first invasive cleavage, since the INVADER and primary probe are supplied in molar excess. In the second invasive cleavage, it is the released flap that is limiting. When these two cleavage reactions are performed sequentially, the fluorescence signal from the composite reaction accumulates linearly with respect to the target RNA amount.

The INVADER invasive cleavage reaction has been shown to be useful in the detection of RNA target strands (See e.g., U.S. Pat. No. 6,001,567, incorporated herein by reference in its entirety). As with the INVADER assay for the detection of DNA (Lyamichev et al., Nat. Biotechnol., 17:292 [1999]), the reactions may be run under conditions that permit the cleavage of many copies of a probe for each copy of the target RNA present in the reaction. In one embodiment, the reaction may be performed at a temperature close to the melting temperature (Tₘ) of the probe that is cleaved, such that the cleaved and uncleaved probes readily cycle on and off the target strand without temperature cycling. Each time a full-length probe binds to the target in the presence of the INVADER oligonucleotide, it may be cleaved by a 5′ nuclease enzyme, resulting in an accumulation of cleavage product. The accumulation is highly specific for the sequence being detected, and may be configured to be proportional to both time and target concentration of the reaction. In another embodiment, the temperature of the reaction may be shifted (i.e., it may be raised to a temperature that will cause the probe to dissociate) then lowered to a temperature at which a new copy of the probe hybridizes to the target and is cleaved by the enzyme. In a further embodiment, the process of raising and lowering the temperature is repeated many times, or cycled, as it is in PCR (Mullis and Faloona, Methods in Enzymology, 155:335 [1987], Saiki et al., Science 230:1350 [1985]). 5′ nucleases of Pol A type DNA polymerases are preferred for cleavage of an invasive cleavage structure that comprises an RNA target strand.

Approaches to designing INVADER assays for the detection of RNA targets can vary depending on the needs of a particular assay. For example, in some embodiments, an RNA to be detected or analyzed may be present in a test sample at low levels, so a high level of sensitivity (i.e., a low limit of detection, or LOD) may be desirable; in other embodiments, an RNA (e.g. NOS2A mRNA) may be abundant, and may not require an especially sensitive assay for detection. In some embodiments, an RNA to be detected may be similar to other RNAs in a sample that are not intended to be detected, so that a high level of selectivity in an assay is desirable, while in other embodiments, it may be desired that multiple similar RNAs be detected in a single reaction, so an assay may be provided that is not selective with respect to the differences among these similar RNAs.

In some embodiments it is especially desirable to avoid detection of any DNA molecules related to the target RNA molecules in a reaction. In some embodiments, this is accomplished by designing INVADER assay probe sets to RNA splice junctions, such that only the properly spliced mRNAs provide the selected target sites for detection. In other embodiments, samples are handled such that DNA remains double stranded (e.g., the nucleic acids are not heated or otherwise subjected to denaturing conditions), and is thus not available to serve as target in an INVADER assay reaction. In other embodiments, cells are lysed under conditions that leave nuclei intact, thereby containing and preventing detection of the genomic DNA, while releasing the cytosolic mRNAs into the lysate solution for detection by the assay.

In some embodiments, the INVADER assay is to be used for detection or quantitation of an entire RNA having a particular variation of a sequence (e.g., a mutation a SNP, a particular spliced junction); in such embodiments, the location of the base or sequence to be detected is a determining factor in the selection of a site for the INVADER assay probe set to hybridize. In other embodiments, any portion of an RNA target may be used to indicate the presence or the amount of the entire RNA (e.g., as in gene expression analysis). In this case, the probe sets may be directed toward a portion of the RNA selected for optimal performance (e.g., sites determined to be particularly accessible for probe hybridization) as a target in the INVADER assay.

II. INVADER Assay Probe Design and Assay Optimization

A discussion of INVADER assay probe design (e.g. for designing probes for NOS2A, SEQ ID NO:1) is divided into the following sections:

i. Target site selection based on accessibility

ii. Target site selection based on selectivity

iii. Oligonucleotide design

a. Target-specific regions: length and melting temperature

b. Non-complementary regions

c. Folding and dimer analysis

iv. Assay performance evaluation

v. Design and assay optimization

i. Target Site Selection Based on Accessibility

One consideration in the selection of sites for detection is the availability of the target site for hybridization of the assay probe set. To simply use randomly selected complementary oligonucleotides for a given RNA target without prior knowledge of regions of the RNA that allow efficient hybridization can be an ineffective approach. For example, it is estimated that targeting RNA with antisense oligonucleotides based on random design results in one out of 18-20 tested oligonucleotides showing significant inhibition of gene expression (Sczakiel, Fronteirs in Biosciences 5:194 [2000]; Patzel et al., Nucleic Acids Res., 27:4328 [1999]; Peyman et al., Biol. Chem. Hoppe-Seyler 367:195 [1995]; Monia et al., Nature Med., 2:668 [1996]). Secondary and tertiary structures of RNA are thought to be the major reasons that influence the ability of an oligonucleotide to bind targeted regions of the RNA (Vickers et al., Nucleic Acids Res., 28:1340 [2000]; Lima et al., Biochemistry 31:12055 [1992]; Uhlenbeck, J. Mol. Biol., 65:25 [1972]; Freier and Tinoco, Biochemistry 14:3310 [1975]). This is due to the hybridization kinetics and thermodynamics of destroying any structural motifs of the RNA and, in return, hybridizing the complementary DNA oligonucleotide (Patzel et al., Nucleic Acids Res., 27:4328 [1999]; Mathews et al., RNA 5:1458 [1999]). Thus, the ability to identify regions of RNA that are “accessible” for hybridization is important for design and selection of effective oligonucleotides.

There are several experimental and theoretical methods available for identifying accessible regions in RNA. These include the use of RNase-H footprinting (Ho et al., Nature Biotechnology 16:59 [1998]; Mateeva et al., Nucleic Acids Res., 25:5010 [1997]; Mateeva et al., Nature Biotechnology 16:1374 [1998]), complementary arrays of oligonucleotide libraries (Southern et al., Nucleic Acids Res., 22:1368 [1994]; Mir and Southern, Nature Biotechnology 17:788 [1999]), ribozyme libraries with random hexamer internal guide sequences (Campbell and Cech, RNA 1:598 [1995]; Lan et al., Science 280:1593 [1998]), and RNA and DNA structure prediction computer programs (Sczakiel, Frontiers in Biosciences 5:194 [2000]; Patzel et al., Nucleic Acids Res., 27:4328 [1999]; Zuker, Science 244:48 [1989]; Walton et al., Biotechnol. Bioeng., 65:1 [1999]). Recently, new methods have been developed that use primer extension to identify sites in RNAs that are accessible for hybridization. Target nucleic acids (e.g., mRNA target nucleic acids) are contacted with a plurality of primers containing a 3′ a region of degenerate sequence and primer extension reactions are conducted. Where the target nucleic acid is an RNA molecule, preferred enzymes for use in the extension reactions are reverse transcriptases, which produce a DNA copy of the RNA template. Folded structures present in the target nucleic acid affect the initiation and/or efficiency of the extension reaction. The extension products of the primers are analyzed to provide a map of the accessible sites. For example, certain extension products are not generated where the primer is complementary to a sequence that is involved in a folded structure. Regions of the target nucleic acid that do not allow hybridization of the primer and do not result in the production of an extension product are considered inaccessible sites. In contrast, the presence of an extension product indicates that the primer was able to bind to an accessible region of the target nucleic acid. Such methods are referred to herein as “reverse transcription with random oligonucleotide libraries” or “RT-ROL” (H T Allawi, et al., RNA 7(2):314-27 [2001], herein incorporated by reference). The use of a physical measurement such as RT-ROL or array hybridization provides the most direct evidence of the accessibility of a site on an RNA strand. In general, INVADER assays directed toward accessible regions produce stronger signals for a given amount of RNA than assays directed toward less accessible regions of an RNA strand. For the detection of rare RNAs (e.g., fewer than about 5,000 to 10,000 copies per INVADER assay reaction), or in any assay wherein it is desirable to have the best (i.e., lowest) limit of detection possible, it may be beneficial to start the assay design by analyzing the RNA structure using RT-ROL or another method of physical analysis.

In other embodiments, ease of assay design may be more important than creation of an assay with a particularly low LOD. Structure prediction software can simplify the task of determining which parts of an RNA are likely to be single stranded, and thus be more accessible for probe hybridization. As first step, the sequence of an RNA to be detected is entered into an electronic file. It may be entered manually or imported from a file (e.g., a sequence data file, or a word processing file). In some embodiments, the sequence is downloaded from a database, such as GenBank or EMBL. The RNA sequence can then be analyzed using a program such as mfold (Zucker, Science 244:48 [1989]), OligoWalk (Mathews et al., RNA 5:1458 [1999]), and variations of both (Sczakiel, Frontiers in Biosciences 5:194 [2000]; Patzel et al., Nucleic Acids Res., 27:4328 [1999]; Walton et al., Biotechnol. Bioeng., 65:1 [1999]).

Mfold Analysis for Target RNA Structure Prediction.

The output of mfold analysis can be used in several ways to assist in identifying accessible regions of an RNA target molecule. In one embodiment, the mfold program is used to generate an “ss count” file for identifying regions least likely to be involved in intra-strand baseparing. In another embodiment, the mfold program is used to generate a “.ct” file, a file used as input information for use with RNA Structure 3.5 to perform an OligoWalk analysis. In preferred embodiments, for either use, the sequence to be detected is entered into mfold. In a preferred embodiment, the settings used in the mfold analysis include:

Folding Temperature: 37° C. (Even though the INVADER reaction may not be conducted at this temperature.)

% Suboptimality: 5

# foldings: 50

Window Parameter: Default

Maximum distance between paired bases: No Limit

Select BATCH folding

Enter: an e mail address where the results are to be sent when ready

Image Resolution: High

Structure Format: Bases

Base Number Frequency: Default

Structure Rotation Angle: 0

Structure Annotation: SS-Count

1M NaCl (Australian mfold Internet site only)

When results are ready, an e mail message is sent containing the Web address of the results. The only file that is necessary for subsequent INVADER assay probe design analysis is the SS-Count file, which is then downloaded from the Results page. An exemplary mfold analysis using a GenBank entry for Human Ubiquitin (#4506712) is shown below:

Oligo Walk Structure prediction With RNA Structure 3.5 for Accessible Sites Identification

In some embodiments, the program OligoWalk, a module of the software “RNAStructure” (Mathews et al., RNA 5:1458 [1999]) is used in the selection of sites that are more likely to be accessible for oligonucleotide binding. OligoWalk uses sets of thermodynamic parameters for both RNA and DNA, and their hybrids (Allawi and SantaLucia, Biochemistry 36:10581 [1997]; Mathews et al., J. Mol. Biol., 288:911 [1999]; Sugimoto et al., Biochemistry 34:11211 [1995]) in an algorithm that relies on mfold for RNA secondary structure prediction (Zucker, Science 244:48 [1989]). OligoWalk is designed to predict the most favorable regions of an RNA target for designing antisense oligonucleotides by estimating the overall thermodynamics of hybridizing an antisense oligomer to the RNA by taking into account the thermodynamics of destroying any structural motifs in the RNA target or the antisense oligonucleotide. The affinity of the oligomer to its target is expressed as an overall Gibbs free energy change of a self-structured oligomer, and of a target associating into an oligomer-target complex. This free energy is usually a negative number, indicating favorable binding, and is expressed in ‘kcal/mol’ units. OligoWalk analysis is performed with 8 to 15 base oligonucleotide size to resemble the average length of the analyte specific region of the Signal Probe. Plotting the total binding energy against the length of the RNA generates a graph of peaks and valleys. The lowest negative values generally indicate the most favorable sites for oligonucleotides to bind. The most inaccessible regions have positive binding energy values, and generally are a poor sites for assay probe design

In a preferred embodiment, the OligoWalk module of RNA Structure 3.5 is used to determine binding energies by performing an 8-base OligoWalk using the following settings:

Break Local Structure

Include suboptimal structures

Oligo Length: 8 nt

Oligo Concentration: 100 nM

Oligo Type: DNA

Walk entire Target RNA

When these parameters have been set, the sequence file to be folded (the “.ct” output file from mfold) can be selected and opened. Once the sequence has been folded, a report can be created using the Output menu. The report is imported into Excel and the data generated above is plotted. In a preferred embodiment, the OligoWalk data is graphed with the SS-Count data. The regions displaying the lowest free energy values (i.e., the largest negative numbers) are generally the most likely to be accessible for hybridization. In preferred embodiments, the 3′ end and the majority of the target-binding region of the probe oligonucleotide complement an accessible region of the target RNA. In particularly preferred embodiments, the majority of the binding site for the corresponding INVADER oligonucleotide falls within the same accessible region. In another preferred embodiment, the binding site for an INVADER oligonucleotide falls within a nearby accessible region.

An INVADER oligonucleotide can generally be positioned to bind to a less accessible site. While not limiting the present invention to any particular mechanism, it is observed that the INVADER oligonucleotides are generally longer than probe oligonucleotides used in the INVADER assay reactions and, because they are generally designed to remain bound to the target at the reaction temperature, they will be selected to have a Tₘs about 12 to 15° C. higher than that of a corresponding probe. Consequently, INVADER oligonucleotides may more readily break the local target structure, and thus may be less dependent on the accessibility of the target-binding site.

In selecting among accessible sites for the design of INVADER assay oligonucleotides, the base composition of the site is also considered. It has been observed that stretched of more than 4 or 5 of the same nucleotide in a row (e.g., . . . AAAA . . . or . . . CCCC . . . ) in any portion of the binding site for the assay oligonucleotides may reduce the performance of the probe set in the assay (e.g., by increasing background or decreasing specificity). Thus, in preferred embodiments, any stretches comprising four or more repeated bases are generally avoided. Another consideration is the effect of base composition on lengths of the oligonucleotides in the probe set. In many cases, targeting A-T rich sequences requires the use of longer oligonucleotides for a reaction performed at a given temperature, compared to the length of oligonucleotides targeted to sequences having a more even distribution of A-T and G-C bases. Longer oligonucleotides can be more prone to formation of intrastrand structures and dimer structures. Thus, it is preferred that the distribution or A-T bases and G-C bases within a target region be as close to even (i.e., about 50% G-C content) as the region to be detected permits. In particularly preferred embodiments, the distribution of A-T and G-C positions is evenly distributed across the binding sites (e.g., not having all A-T positions in one half, with all G-C positions in the other).

ii. Target Site Selection Based on Selectivity

In some embodiments, probe sets are designed to examine highly homologous, or closely related RNA targets (i.e., targets that are very similar in sequence). In such embodiments, the RNA or homologous cDNA sequences are compared, e.g., using an alignment program such as MEGALIGN (DNAstar Madison, Wis.).

In some embodiments, selectivity is provided by designing probe sets to detect splice junctions. Splice junctions can be identified by aligning the cDNA and gene sequences using an alignment program (e.g. MEGALIGN) or under the BLAST menu at the NCBI website (BLAST 2 sequences). Splice junctions are also often listed in the GenBank report (intron/exon sites). INVADER assay oligonucleotide sets are designed such that the probe and INVADER oligonucleotides are complementary to the coding strand (mRNA), generally with the cleavage site being as close to the splice junction as possible. In some embodiments, different splice junctions within an mRNA are analyzed for accessibility, as described above. In preferred embodiments, probe sets are designed to detect one or more splice junctions showing greater accessibility compared to the accessibility of other splice junctions within the same RNA target.

In some embodiments designed to exclude detection of RNAs related to the target RNA, sequences are examined to identify bases that are unique to the target RNA when compared to the other similar sequences from which the target is distinguished. Generally, the unique base is positioned to hybridize to the 5′ end of the target-specific region of the probe oligonucleotide. In some embodiments, two adjacent bases are unique to the target compared to the related RNA. If two adjacent unique bases are available in an appropriately accessible portion of the target RNA, it is preferred that these bases be used as the site around which the probe and INVADER oligonucleotides sets are designed. In some embodiments, the two unique bases are positioned such that the site of cleavage of the probe is between the two base-pairs they form with the probe. In other embodiments, one of the unique bases is in the last position of the hybridization site of the INVADER oligonucleotide (i.e., it is positioned to base-pair to the penultimate residue on the 3′ end of the INVADER oligonucleotide).

In some embodiments, the assay is designed to include detection of RNAs that are similar, but not identical, to the target RNA. If the assay is being designed for inclusive detection, the compared sequences are examined to identify sites having complete homology. Such designs can be created to detect homologous sequences in the same species or between species. Generally, the most homologous regions are selected as hybridization targets for probe oligonucleotides. Generally, some variation can be tolerated, for example, if it is not at the base that would hybridize to the 5′ end of the target-specific region of a probe. In some embodiments, variation is accommodated by the use of degenerate bases in the INVADER assay oligonucleotides (e.g., mixtures of bases are used at positions within thesynthesized probe, INVADER and/or stacker oligonucleotides, the mixtures selected to complement the mixture of specific bases present in the collection of related target RNAs).

iii. Oligonucleotide Design

a. Target-Specific Regions: Length and Melting Temperature

As described above in Section I (a) concerning the oligonucleotide design, in some embodiments, the length of the analyte-specific regions are defined by the temperature selected for running the reaction. Starting from the desired position (e.g., a variant position or splice junction in a target RNA, or a site corresponding to a low free energy value in an OligoWalk analysis) an iterative procedure is used by which the length of the ASR is increased by one base pair until a calculated optimal reaction temperature (Tₘ plus salt correction to compensate for enzyme and any other reaction conditions effects) matching the desired reaction temperature is reached. In general probes are selected to have an ASR with a calculated Tₘ of about 60° C. if a stacking oligonucleotide is not used, and a Tₘ of about 50 to 55° C. if a stacking oligonucleotide is used (a stacking oligonucleotide typically raises the Tₘ of a flanking probe oligonucleotide by about 5 to 15° C.). If the position of variation or a splice junction is a starting position, then the additions are made to the 3′ end of the probe. Alternatively, if the 3′ end of the probe is to be positioned at the most accessible site, the additions are in the 5′ direction. In some embodiments, wherein a stacker oligonucleotide is to be used, it is preferred that the probe be designed to have a 3′ base that has stable stacking interaction interface with the 5′ base of the stacker oligonucleotide. The stability of coaxial stacking is highly dependent on the identity of the stacking bases. Overall, the stability trend of coaxial stacking in decreasing order is purine:purine>purine:pyrimidine≈pyrimidne:purine>pyrimidine:pyrimidine. In other embodiments employing a stacker, a less stable stacking interaction is preferred; in such cases the probe 3′ base and/or the stacker 5′ base are selected to provide a leass stable stacking interaction. In some embodiments, the probe 3′ base and/or the stacker 5′ base are selected to have a mismatch with respect to the target strand, to reduce the strength of the stacking interaction.

The same principles are also followed for INVADER oligonucleotide design. Briefly, starting from the position N, additional residues complementary to the target RNA starting from residue N−1 are then added in the upstream direction until the stability of the INVADER-target hybrid exceeds that of the probe (and therefore the planned assay reaction temperature). In preferred embodiments, the stability of the INVADER-target hybrid exceeds that of the probe by 12-15° C. In general, INVADER oligonucleotides are selected to have a Tₘ near 75° C. Software applications, such as INVADERCREATOR (Third Wave Technologies, Madison, Wis.) or Oligonucleotide 5.0 may be used to assist in such calculations.

If a stacking oligonucleotide is to be used, similar design principles are applied. The stacking oligonucleotide is generally designed to hybridize at the site adjacent to the 3′ end of the probe oligonucleotide, such that the stacker/target helix formed can coaxially stack with the probe/target helix. The sequence is selected to have a calculated Tₘ of about 60 to 65° C., with the calculation based on the use of natural bases. However, stacking oligonucleotides are generally synthesized using only 2′-O-methyl nucleotides, and consequently, have actual Tₘs that are higher than calculated by about 0.8° C. per base, for actual Tₘs close to 75° C.

In some embodiments, ARRESTOR oligonucleotides are included in a secondary reaction. ARRESTOR oligonucleotides are provided in a secondary reaction to sequester any remaining uncleaved probe from the primary reaction, to preclude interactions between the primary probe and the secondary target strand. ARRESTOR oligonucleotides are generally 2′-O-methylated, and comprise a portion that is complementary to essentially all of their respective probe's target-specific region, and a portion that is complementary to at least a portion of the probe's flap regions (e.g., six nucleotides, counted from the +1 base towards the 5′ end of the arm).

b. Non-Complementary Regions

Probe 5′ Arm Selection

The non-complementary arm of the probe, if present, is preferably selected (by an iterative process as described above) to allow the secondary reaction to be performed at a particular reaction temperature. In the secondary reaction, the secondary probe is generally cycling, and the cleaved 5′ arm (serving as an INVADER oligonucleotide) should stably bind to the secondary target strand.

INVADER Oligonucleotide 3′ Terminal Mismatch Selection

In preferred embodiments, the 3′ base of the INVADER oligonucleotide is not complementary to the target strand, and is selected in the following order of preference (listed as INVADER oligonucleotide 3′ base/target base):

| | |
|---|---|
| C in target: | C/C > A/C > T/C > G/C |
| A in target: | A/A > C/A > G/A > T/A |
| G in target: | A/G > G/G > T/G > C/G |
| U in target: | C/U > A/U > T/U > G/U |

c. Folding and Dimer Analysis

In some embodiments, the oligonucleotides proposed for use in the INVADER assay are examined for possible inter- and intra-molecular structure formation in the absence of the target RNA. In general, it is desirable for assay probes to have fewer predicted inter- or intra molecular interactions. In some embodiments, the program OLIGO (e.g., OLIGO 5.0, Molecular Biology Insights, Inc., Cascade, Colo.) is used for such analysis. In other embodiments, the program mfold is used for the analysis. In yet other embodiments, the RNAStructure program can be used for dimer analysis. The following sections provide stepwise instructions for the use of these programs for analysis of INVADER assay oligonucleotides.

OLIGO 5.0 Analysis for Probe Structure and Interaction Prediction.

Analysis of INVADER oligonucleotides using OLIGO 5.0 comprises the following steps. All menu choices are shown in UPPER CASE type.

1. Launch OLIGO 5.0 and open a sequence file for each mRNA to be analyzed. This is done by using a menu to select the following

Choose FILE->NEW

Paste in longest available sequence

Choose ACCEPT & QUIT (F6)

2. Set Program settings to default

Choose FILE->RESET->ORIGINAL DEFAULTS

3. Identify Probe Oligonucleotide

Select OLIGO LENGTH to be around 16 nucleotides (open the menu for this option by using ctrl-L keystrokes).

Move the cursor indicating the 5′ end of the Current Oligo until the 3′ end is located at the candidate cleavage site residue.

Choose ANALYSE->DUPLEX FORMATION->CURRENT OLIGO (ctrl-D) for a rough determination of the extent of dimer and hairpin formation.

Confirm length of analyte region corresponds with desired reaction temperature [e.g., through the use of Tₘ calculation as described in the Optimization of Reaction Conditions, I (c) of the Detailed Description of the Invention]

Select the “LOWER” button in OLIGO 5.0 to copy the anti-sense sequence (this will be the analyte-specific region of the actual probe oligonucleotide and is anti-sense to the RNA strand.)

Import into a database file.

Save to computer memory.

4. Identify INVADER Oligonucleotide

Choose sequence adjacent to the probe oligonucleotide identified from step 3.

Select OLIGO LENGTH to ˜24 nucleotides

Confirm length of analyte region corresponds with desired reaction temperature [e.g., through the use of Tₘ calculation as described in the Optimization of Reaction Conditions, I (c) of the Detailed Description of the Invention, about 75° C. for INVADER oligonucleotides). Select the “LOWER” button in OLIGO 5.0 to copy the corresponding anti-sense sequence (this will be the analyte-specific region of the actual INVADER oligonucleotide.)

Import into a database file.

Save to computer memory.

5. Addition of Cleaved Arm Sequence and INVADER Oligonucleotide Mismatch Sequence.

Export the Probe oligonucleotide as Upper Primer.

Export the INVADER oligonucleotide as Lower Primer.

EDIT UPPER PRIMER to add in a candidate arm sequence (selected, for example, as described above).

Check that the arm sequence does not create new secondary structures (analysis performed as described above).

EDIT LOWER PRIMER to add in the 3′ mismatched nucleotide that will overlap into the cleavage site (selected according to the guidelines for this mismatched bases, provided above).

Select all Upper and Lower Primer boxes in the “Print/Save Options”

PRINT ANALYSIS of Upper (Probe) and Lower (INVADER) oligonucleotides and check for lack of stable secondary structures.

Save both mRNA sequence and oligonucleotide sequence database files before quitting the program.

Generally, oligonucleotides having detected intra-molecular formations with stabilities of less than −6 ΔG are preferred. Less stable structures represent poor substrates for CLEAVASE enzymes, and thus cleavage of such structures is less likely to contribute to background signal. Probe and INVADER oligonucleotides having less affinity for each other are more available to bind to the target, ensuring the best cycling rates.

The Tₘ of dimerized probes (i.e., probes wherein one probe molecule is hybridized to another probe molecule) should ideally be lower than the Tₘ for the probe hybridized to the target, to ensure that the probes preferentially hybridize to the target sequence at the elevated temperatures at which INVADER assay reactions are generally conducted. Similarly, the Tₘ for the INVADER oligonucleotide hybridized either to itself or to a probe molecule should be lower than the INVADER oligonucleotide/target Tₘ. It is preferred that dimer Tₘs (i.e., Probe/Probe and Probe/INVADER oligonucleotide) be 25° C. or less to ensure that they will be unlikely to form at the planned reaction temperature.

The melting temperatures for each of these complexes can be determined as described above in Optimization of Reaction Conditions, I (c) of the Detailed Description of the Invention, or by using the OLIGO software. Once RNAs sites and several candidate INVADER assay oligonucleotide sets are selected according to the process outlined above, the candidate oligonucleotide sets can be ranked according to the degree to which they comply with preferred selection rules, e.g., their location on the SS-Count average plot (peak, valley, neither), and the energetic predictions of probe and INVADER oligonucleotide interactions. In some embodiments, the ranked probe sets are tested in order of rank to identify one or more sets having suitable performance in an RNA INVADER assay. In other embodiments, several of the top ranked sets (e.g., two, three or more) are selected for testing, to rapidly identify one or more sets having suitable or desireable performance.

Mfold Analysis for Probe Structure and Interaction Prediction

Analysis of probe and INVADER oligonucleotide interactions may be performed using mfold for DNA provided by Michael Zuker, available through Rensselaer Polytechnic Institute at bioinfo.math.rpi.edu/˜mfold/dna/form1.cgi. The analysis is performed without changing the default ionic conditions, and with a selected temperature of 37° C. and with % suboptimality set to 75. Each sequence (e.g., probe, INVADER oligonucleotide, stacker, etc.) is folded using the program to check for any unimolecular structure formation (e.g., hairpins). The energies provided by mfold gives for unimolecular structures can be used as provided, without further calculations.

Bimolecular structure formation for a given oligonucleotide is assessed by typing in the oligonucleotide sequence (5′ to 3′) followed by the sequence of a small, stable hairpin forming sequence (e.g., CCCCCTTTTGGGGG [SEQ ID NO:707]), followed by the same oligonucleotide sequence, again listed 5′ to 3. Constraints are entered to require that these Ts remain single-stranded and the strings of Cs and Gs in this spacer are basepaired. The command “F” is used to force basepairing, while the command “P” is used to prohibit basepairing, and the positions of the forced or prohibited basepairs are counted from the 5′ end. For example, if the sequence of interest is a 20-mer, then the following is entered:

F 21 0 5 [this forces the C's, C21 to C25, to base pair]

P 26 0 4 [this forces the T's, T26 to T29, to be single stranded]

F 30 0 5 [this forces the G's, G30 to G34, to base pair]

On examination of the resulting structures, the stability of each can be estimated by subtracting the stability (i.e., the thermodynamic measures) of the central spacer hairpin from the total result (i.e., Thermodynamics of possible structure=mfold structure thermodynamics−core hairpin thermodynamics). For convenience, in some embodiments, any nearest neighbor interactions between the central hairpin and dimers formed by the test sequence are ignored for this calculation; a more accurate analysis would require consideration of this interaction. The core hairpin formed by CCCCCTTTTGGGGG (SEQ ID NO:707) has the following thermodynamics: G=−5.3; H=−37.8; S=−104.8.

The process can be demonstrated using the following probe sequence: 5′-CCCTATCTTTAAAGTTTTTAAAAAGTTTGA-3′ (SEQ ID NO:708). The oligonucleotide sequence is examined by mfold analysis for bimolecular structures using the following steps.

1—In mfold Sequence Box Type:

| | |
|---|---|
| CCCTATCTTTAAAGTTTTTAAAAAGTTTGACCCC | (SEQ ID NO:137) |
| CTTTTGGGGGCCCTATCTTTAAAGTTTTTAAAAA | |
| GTTTGA | |

2—In the Constraint Box Type:

P 3604

F 31 0 5

F 40 0 5

Results (Showing One):

```
Structure 1
dG = −14.2 dH = −150.5 dS = −439.5 Tm = 69.3
CCCTATCTTT |G G -------- T
                AAA TTTTTAAAAA TTTGA CCCCCT
                TTT AAAAATTTTT AAATT GGGGGT
--------AG \hat{ }G G TCTATCCC T
```

To Evaluate the stability of the Duplex:

```
CCCTATCTTT |G G
                AAA TTTTTAAAAA TTTGA
                TTT AAAAATTTTT AAATT
--------AG \hat{ }G G TCTATCCC
```

the thermodyanamic values for the hairpin alone are subtracted from the values for the complete structure:

G=−14.2−(−5.3)=−8.9,

H=−150.5−(−37.8)=−112.7,

S=−439.5−(−104.8)=−334.7,

Using a calculation wherein Tₘ (° C.)={H/[S+R ln (CT/4)]}−273.15, wherein R is the gas constant 1.987 (cal/K.mol), ln is the natural log, and CT is the total single strand concentration in Molar, this results in a calculated Tₘ of 46.1° C. for the non-hairpin portion of the structure.

The above method is not limited to the use of the core hairpin sequence CCCCCTTTTGGGGG but rather any stable hairpin sequences can be used. For example, CGCGCGGAACGCGCG (SEQ ID NO:138) or CCCGGGTTTTCCCGGG (SEQ ID NO:139). However, if a different hairpin sequence is used, one needs to calculate its stability using mfold and use its thermodynamics in the subsequent calculations.

RNAStructure for Oligonucleotide Interaction Prediction

Dimer formation can also be evaluated using the RNAStructure program. Unlike mfold, RNAStructure allows the calculation of all possible oligonucleotide-oligonucleotide interactions and provides an output .ct file. One can then view the structures using any .ct viewing program such as RNAStructure or RNAvis (1997, P. Rijk, University of Antwerp (UIA), available on the Internet at rrna.uia.ac.be/rnavis) and evaluate the stability of any dimer formation using the nearest-neighbor model (Borer et al., 1974) and DNA nearest-neighbor parameters (Allawi & SantaLucia, 1997).

For example, to evaluate the propensity of the sequence 5′ AGGCGCACCAATTTGGTGTT 3′ (SEQ ID NO:140) for dimer formation using the DNA Fold Intermolecular module of RNAStructure, the sequence is saved into a file (e.g., probe.seq) and the following parameters are set:

Sequence file 1: probe.seq

Sequence file 2: probe.seq

CT file: dimer.ct

Max % Energy difference: 50

Max number of structures: 20

Window size: do not change

After the calculation is done, one can view the resulting .ct file using the “view” module of RNAStructure. Generally, there will be several structures within the .ct file. The view module is used to view them individually. One of the dimers that the test sequece, above, can form according to RNAStructure is:

```
AGGCG TT
    CACCAATTTGGTG
    GTGGTTTAACCAC
  TT GCGGA
```

According to the nearest-neighbor model (i.e., using DNA nearest-neighbor and mismatch parameters [Allawi & SantaLucia, 1997]), the stability of this duplex in 1M NaCl and at a probe concentration of 100 M is:

G°₃₇=−10.07

H=−87.6

S=−250.1

Tm=50.1° C.

By changing the identities of Sequence Files 1 & 2, RNAStructure can be used to evaluate the possibility of any dimer formation between pairs of all of the DNA oligonucleotides present in an INVADER assay reaction.

iv. Assay Performance Evaluation

Probe sets selected according to the guidelines provided above can be tested in the INVADER assay to evaluate performance. The bi-plex embodiments (configured for assaying both a test gene and an internal reference gene) may also be analyzed such that both reactions function without interfering with each other. While the oligonucleotides are designed to perform at or near a particular desired reaction temperature, the best performance for a given design may not be precisely at the intended temperature. Thus, in evaluating any new INVADER assay probe set, it can be helpful to examine the performance in the INVADER assay conducted at several different reaction temperatures, over a range of about 10 to 15° C., centered around the designed temperature. For convenience, temperature optimization can be performed on a temperature gradient thermocycler with a fixed amount of RNA (e.g., 2.5 amoles of an in vitro transcript per reaction), and for a fixed amount of time (e.g., 1 hour each for Primary and Secondary reactions). The temperature gradient test will reveal the temperature at which the designed probe set produces the best performance (e.g., the highest level of target-specific signal compared to background signal, generally expressed as a multiple of the zero-target background signal, or “fold over zero”).

The results can be examined to see how close the measured temperature optimum is to the intended temperature of operation. In some embodiments, it is desirable to have probe sets that operate at or near a pre-selected temperature. If the measured temperature optimum is higher than the desired reaction temperature, a probe design can be altered in ways that tend to reduce the probe/target Tₘ (e.g., shortened by one or more bases, or altered to contain one or more mismatched bases). In some embodiments, wherein a stacker oligonucleotide is not used, wherein the reaction temperature is more than 7° C. above the desired reaction temperature, and wherein the performance (e.g., the fold over zero) is acceptable, use of a 3′ mismatch on the probe oligonucleotide is likely to lower the reaction temperature without otherwise altering the assay performance.

An LOD determination can be made by performing reactions on varying amounts of target RNA (e.g., an in vitro transcript control RNA of known concentration). In preferred embodiments, a designed assay has an LOD of less than 0.05 attomole. In particularly preferred embodiments, a designed assay has an LOD of less than 0.01 attomole. It is contemplated that the same guideline provided above for reducing the LOD of a designed assay may be used for the purpose of raising the LOD of a designed assay, i.e., to make it LESS sensitive to the presence of a target RNA. For example, it may be desirable to detect an abundant RNA and a rare RNA in the same reaction. In such a reaction, it may be desirable to attenuate the signal generated for the abundant RNA so that it does not overwhelm the signal from the rarer species. In some embodiments this may be done by designing probe sets for reduced signal generation, e.g., an LOD of at least (not less than) 0.5 attomoles. In some embodiments, a single step INVADER assay may be used for detection of abundant targets in a sample, while sequential INVADER reactions to amplify signal, as described in Section II, may be used for less abundant analytes in the same sample. In preferred embodiments, the single step and the sequential INVADER assay reactions for the different analytes are performed in a single reaction.

In some embodiments, time course reactions are run, wherein the accumulation of signal for a known amount of target is measured for reactions run for different lengths of time. This measurement will establish the linear ranges, i.e., the ranges in which accurate quantitative measurements can be made using a given assay design, with respect to time and starting target RNA level.

v. Design and Assay Optimization

Some designed assays may not meet the preferred performance criteria described above. A number of variations on the performance of INVADER assay reactions have been described herein. In optimizing performance of the INVADER assay for the detection of RNA targets, these variations may be used alone or in combination. For example, in some embodiments, a stacker oligonucleotide is employed. Also for example, in some embodiments, a biplex assay is employed where both a target gene and an internal reference gene are to be detected in the same well. While not limiting the present invention to any particular mechanism of action, in some embodiments, a stacker oligonucleotide may enhance performance of an assay by altering the hybridization characteristics (e.g., Tₘ) of a probe or an INVADER oligonucleotide. In some embodiments, a stacker oligonucleotide may increase performance by enabling the use of a shorter probe. In other embodiments, a stacker oligonucleotide may enhance performance by altering the folded structure of the target nucleic acid. In yet other embodiments, the enhancing activity of the stacker oligonucleotide may involve these and other mechanisms in combination.

In other embodiments, the target site may be shifted. In some embodiments, reactions are optimized by testing multiple probe sets that shift along a suspected accessible site. In preferred embodiments, such probe sets shift along the accessible site in one to two base increments. In embodiments wherein accessible sites have previously been predicted only by computer analysis, physical detection of the accessible sites may be employed to optimize a probe set design. In preferred embodiments, the RT-ROL method of detecting accessible sites is employed. In some embodiments, optimization of a probe set design may require shifting of the target site to a newly identified accessible site.

In some embodiments, e.g., wherein an accessible site has been identified yet probe set performance is low, a change in the design of a probe 5′ arm may improve assay performance without altering the site targeted. In other embodiments, altering the length of an ARRESTOR oligonucleotide (e.g., increasing the length of the portion that is complementary to the 5′ arm region of the probe) may reduce background signal, thus increasing the probe stet performance.

Other variations on oligonucleotide design may be employed to alter performance in an assay. Some modifications may be employed to shift the ideal operating temperature of a probe set design into a preferred temperature range. For example, the use of shorter oligonucleotides and the incorporation of mismatches generally act to reduce the Tₘs, and thus reduce the ideal operating temperatures, of designed oligonucleotides. Conversely, the use of longer oligonucleotides and the employment of stacking oligonucleotides generally act to increase the Tₘs, and thus increase the ideal operating temperatures of the designed oligonucleotides.

Other modifications may be employed to alter other aspects of oligonucleotide performance in an assay. For example, the use of base analogs or modified bases can alter enzyme recognition of the oligonucleotide. In some embodiments, such modified bases are used to protect a region of an oligonucleotide from nuclease cleavage. In other embodiments, modified bases are used to affect the ability of an oligonucleotide to participate as a member of a cleavage structure that is not in a position to be cleaved (e.g., to serve as an INVADER oligonucleotide to enable cleavage of a probe). These modified bases may be referred to as “blocker” or “blocking” modifications. In some embodiments, assay oligonucleotides incorporate 2′-O-methyl modifications. In other embodiments, assay oligonucleotides incorporate 3′ terminal modifications (e.g., NH₂, 3′ hexanol, 3′ phosphate, 3′ biotin).

In yet other embodiments, the components of the reaction may be altered to affect assay performance. For example, oligonucleotide concentrations may be varied. Oligonucleotide concentrations can affect multiple aspects of the reaction. Since melting temperatures of complexes are partly a function of the concentrations of the components of the complex, variation of the concentrations of the oligonucleotide components can be used as one facet of reaction optimization. In the methods of the present invention, ARRESTOR oligonucleotides may be used to modulate the availability of the primary probe oligonucleotides in an INVADER assay reaction. In some embodiments, an ARRESTOR oligonucleotide may be excluded. Other reaction components may also be varied, including enzyme concentration, salt and divalent ion concentration and identity.

III. Kits for Performing the RNA INVADER Assay

In some embodiments, the present invention provides kits comprising one or more of the components necessary for practicing the present invention. For example, the present invention provides kits for storing or delivering the enzymes of the present invention, one or more nutraceutical compounds, lysis buffer, and/or other reaction components necessary to practice a cleavage assay (e.g., the INVADER assay). The kit may include any and all components necessary or desired for the enzymes or assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.). In some embodiments one or more the reaction components may be provided in a predispensed format (i.e., premeasured for use in a step of the procedure without re-measurement or re-dispensing). In some embodiments, selected reaction components are mixed and predispensed together. In preferred embodiments, predispensed reaction components are predispensed and are provided in a reaction vessel (including but not limited to a reaction tube or a well, as in, e.g., a microtiter plate). In particularly preferred embodiments, predispensed reaction components are dried down (e.g., desiccated or lyophilized) in a reaction vessel.

Additionally, in some embodiments, the present invention provides methods of delivering kits or reagents to customers for use in the methods of the present invention. The methods of the present invention are not limited to a particular group of customers. Indeed, the methods of the present invention find use in the providing of kits or reagents to customers in many sectors of the biological and medical community, including, but not limited to customers in academic research labs, customers in the biotechnology and medical industries, and customers in governmental labs. The methods of the present invention provide for all aspects of providing the kits or reagents to the customers, including, but not limited to, marketing, sales, delivery, and technical support.

In some embodiments of the present invention, quality control (QC) and/or quality assurance (QA) experiments are conducted prior to delivery of the kits or reagents to customers. Such QC and QA techniques typically involve testing the reagents in experiments similar to the intended commercial uses (e.g., using assays similar to those described herein). Testing may include experiments to determine shelf life of products and their ability to withstand a wide range of solution and/or reaction conditions (e.g., temperature, pH, light, etc.).

In some embodiments of the present invention, the compositions and/or methods of the present invention are disclosed and/or demonstrated to customers prior to sale (e.g., through printed or web-based advertising, demonstrations, etc.) indicating the use or functionality of the present invention or components of the present invention. However, in some embodiments, customers are not informed of the presence or use of one or more components in the product being sold. In such embodiments, sales are developed, for example, through the improved and/or desired function of the product (e.g., kit) rather than through knowledge of why or how it works (i.e., the user need not know the components of kits or reaction mixtures). Thus, the present invention contemplates making kits, reagents, or assays available to users, whether or not the user has knowledge of the components or workings of the system.

Accordingly, in some embodiments, sales and marketing efforts present information about the novel and/or improved properties of the methods and compositions of the present invention. In other embodiments, such mechanistic information is withheld from marketing materials. In some embodiments, customers are surveyed to obtain information about the type of assay components or delivery systems that most suits their needs. Such information is useful in the design of the components of the kit and the design of marketing efforts.

IV. The INVADER Assay RNA Targets

The following section provides a few illustrative examples of mRNAs that may be detected or measured using the methods, compositions and systems of the present invention.

Target Gene mRNA

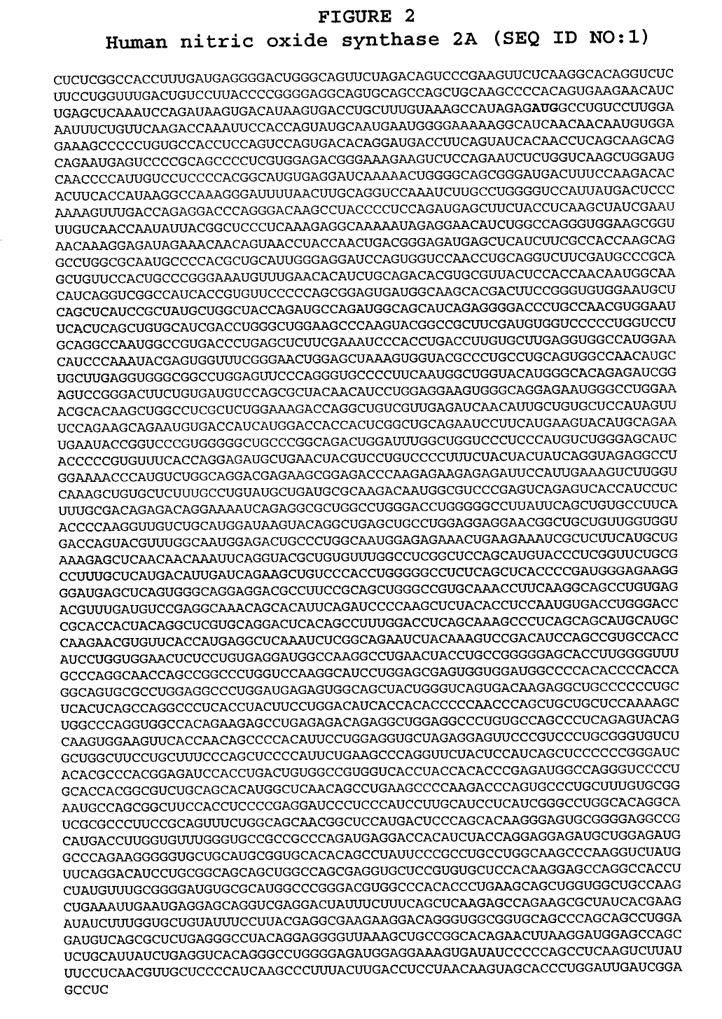

Any mRNA who's expression may be effected by a nutracutical may be detected in the assays of the present invention. For example, human nitric oxide synthase 2A (SEQ ID NO:1, see FIG. 2) may be detected in the nutracutical screening assays of the present invention. Inducible nitric oxide synthase (iNOS or NOS2) is expressed in several cell types including macrophages, endothelial cells, and hepatocytes. In most cells, NOS2 is expressed only after induction by different stimuli such as cytokines. After induction the translated NOS2 enzyme produces high amounts of nitric oxide (NO). Macrophages use the high production of NO to kill bacteria, fungi, virus, parasites and tumor cells. Besides this important immune system function, NO seems to protect against liver injury and is important for skin wound healing. Overexpression of NO is causally involved in diabetes type I, rheumatoid arthritis, multiple sclerosis, inflammatory bowel disease, asthma, and septic shock.

Internal Reference mRNAs (e.g. Housekeeping Controls)

RNAs that are generally present in predicable or invariant amounts in test samples provide useful control targets for detection assays. These controls can be useful in several ways, including but not limited to providing confirmation of the proper function of an assay, and as a standard against which a test result for another RNA can be compared or measured to aid in interpretation of a result. mRNAs for the following genes find particular use in the methods of the present invention.

Human Ubiquitin and Mouse/Rat Ubiquitin

The ubiquitin system is a major pathway for selective protein degradation. Degradation by this system is instrumental in a variety of cellular functions such as DNA repair, cell cycle progression, signal transduction, transcription, and antigen presentation. The ubiquitin pathway also eliminates proteins that are misfolded, misplaced, or that are in other ways abnormal. This pathway requires the covalent attachment of ubiquitin (E1), a highly conserved 76 amino acid protein, to defined lysine residues of substrate proteins.

Human, Rat and Mouse Glyceraldehyde-3-phosphate Dehydrogenase (GAPDH)

GAPDH is an important enzyme in the glycolysis and gluconeogenesis pathways. This homotetrameric enzyme catalyzes the oxidative phosphorylation of D-glyceraldehyde-3-phosphate to 1,3-diphosphoglycerate in the presence of cofactor and inorganic phosphate. A variety of diverse biological properties of GAPDH have been reported. These include functions in endocytosis, mRNA regulation, tRNA export, DNA replication, DNA repair, and neuronal apoptosis.

V. High Throughput Screening

The present invention also contemplates all of the above screening assays (and variations of these assays) in a high throughput format. The high throughput adaptation of these assays will be apparent to those skilled in the art (See, U.S. Pat. No. 5,623,051, and Burbaum et al, 1:72-78, Curr. Opin. Chem. Biol. (1997)). High throughput assays are particularly useful in the present invention because of the ability to screen hundreds, thousands, and even millions of nutraceutical compounds in a short period of time. Typically, standard assays are miniaturized and automated. An example of miniaturization involves replacing a standard 96-well plate with a 1536-well plate which has much smaller wells. A typical number of compounds that may be screened per day is on the order of around, 200 per day. Therefore, high through-put screening is a useful tool when combined with the assays of the present invention in screening nutraceutical compounds to identify those with true beneficial properties (e.g. those that support would support a structure/function claims under the 1994 Dietary Supplement Health and Education Act.

In preferred embodiments, a high throughput system, (e.g. for human iNOS mRNA) is to design the oligonucleotide sets for the iNOS mRNA sequence as well as the internal standard sequence (e.g. Ubiquitin) that function optimally in the INVADER assay under uniform reaction conditions, including temperature, so that all reactions can be carried out in a single microtiter plate. More specifically, assays are configured such that the internal standard can be assayed in the same reaction well as the test gene mRNA (e.g. iNOS sequence). This biplex format involves the construction of two distinct secondary reaction/signal templates and primary probe 5′ flaps that do not interfere with one another. The fluorescent signals from these two different secondary reaction templates should not overlap. An example of such a pair of dyes that meet this criteria are: FAM and red dye (Z38) developed by Epoch Pharmaceuticals (Bothell, Wash.). Both reporter dyes are quenched by a dark quencher (Z28) also developed by Epoch Pharmaceuticals.

VI. Dietary Supplement Health and Education Act

In 1994, the Dietary Supplement Health and Education Act (DSHEA) was passed by Congress and signed into law by President Clinton. Among other provisions, DSHEA added section 201(ff) of the Federal Food, Drug, and Cosmetic Act (FDCA) which defines the term “dietary supplement” as:

(1)(A) A vitamin;

(B) a mineral;

(C) an herb or other botanical;

(D) an amino acid;

(E) a dietary substance for use by man to supplement the diet by increasing the total dietary intake; or

(F) a concentrate, metabolite, constituent, extract, or combination of any ingredient described in clause (A), (B), (C), (D), or (E).

Section 201(ff) additionally requires that a dietary supplement be intended for ingestion in tablet, capsule, powder, softgel, gelcap, or liquid form; or, if not in such form, it should not be represented for use as a conventional food, or as a sole item of a meal or of the diet, and in any event should be labeled as a dietary supplement.

DSHEA also added section 403(r)(6) to the FDCA. Section 403(r)(6) states that for purposes of the requirements for health claims, a statement may be made on a nutritional supplement's labeling if: 1) the statement claims a benefit related to a classical nutrient deficiency disease and discloses the prevalence of such disease in the United States; 2) describes the role of a nutrient or dietary ingredient intended to affect the structure or function in humans; 3) characterizes the documented mechanism by which a nutrient or dietary ingredient acts to maintain such structure or function; or 4) describes general well-being from consumption of a nutrient or dietary ingredient.

Although these statements were referred to as “statements of nutritional support,” FDA no longer uses this term and now refers to these statements as structure/function claims. Section 403(r)(6) authorizes manufacturers complying with a disclaimer statement and a post-market notification procedure to include such structure/function claims in dietary supplement labeling. The nutraccutical testing methods of the present invention may be used to support such structure/function claims by providing the necessary data.

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); DS (dextran sulfate); and ° C. (degrees Centigrade).

### EXAMPLE 1

### Detecting GAPDH mRNA in Cell Lysates

This example describes the detection of human GAPDH in heated cell lysates. Since this gene is constitutively expressed at high levels in cells, expression levels were modulated by varying cell seeding densities. The INVADER assay oligonucleotides for GAPDH were designed to span a splice junction to ensure that the INVADER oligonucleotide and signal probe oligonucleotide hybridize to mature mRNA, and not genomic DNA. Subsequent experiments performed with probes sets that are designed to exonic regions showed that there was no cross-detection of genomic DNA under the cell lysate INVADER reaction (described below). This is primarily due to the observation that the same oligonucleotide sets have temperature optima that are approximately 10-12 degrees Celsius different when used on RNA or DNA.

A MG-63 human fibroblast cell line was seeded at 2×10³-4×10⁵ cells/well in a 96-well tissue culture plate. After 4 hours, the medium was removed and the adherent cells were washed 1×with PBS. The cell were lysed with 32 μl of lysis buffer (0.5% NP-40, 5 mM MgCl₂, 20 mM Tris-Cl, pH 7.5, and 200 μg/ml tRNA) for 5 minutes, and then 8 μg of lysates were transferred to a 96-well polypropylene microtiter plate. The lysates were heated at 90 degrees Celsius for 15 minutes, cooled to room temperature and then 12 μl of primary INVADER reaction mix was added. After a 2 hour incubation at 58 degrees Celsius, 5 μl of secondary reaction mix was added and the reactions incubated for an additional 1 hour at 58 degrees Celsius.

A standard curve was generated (not shown) using a GAPDH in vitro transcript RNA. The in vitro transcript RNA INVADER reactions contained 8 μl of lysis buffer to mimic the conditions of the cell lysates. Previously, it was determined that the cell lysis buffer did not inhibit the INVADER reaction. The amole levels of GAPDH in the cell lysates were calculated from the standard curve. These results indicate that GAPDH can be detected in as few as 500 cells using the INVADER squared FRET assay.

### EXAMPLE 2

### Assaying aP2 mRNA Expression Levels in Cells Exposed to Various Compounds

This example describes assaying aP2 mRNA expression levels in cells exposed to various compounds using an INVADER assay. In particular, the INVADER assay was employed to quantitate mRNAs directly from the cell lysate of a murine 3T3L1 fibroblast cell line. When cultured in the presence of insulin, these 3T3L1 fibroblasts differentiate into adipocytes and concomitantly induce expression of aP2 mRNA. The ability of pharmocolgical compounds to affect the rate and extent of differentiation, for example, is one indicator of their effectiveness in treating Type II diabetes.

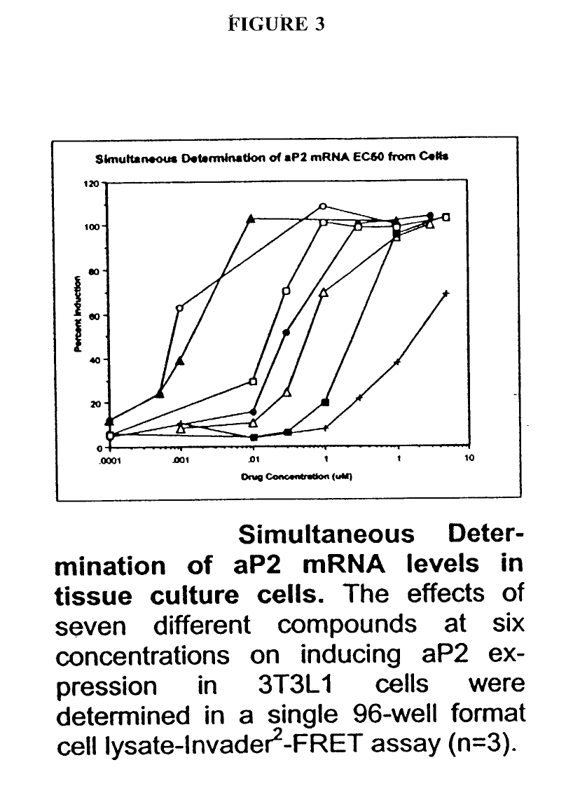

We assayed aP2 mRNA expression in 3T3L1 cells treated with seven different compounds at six different concentrations, along with vehicle controls on a single 96-well tissue culture plate. Assays were conducted with INVADER squared assay, which includes the FRET cassette (see, e.g., FIG. 1A). Within 4 hours of removing the cells from the incubator, using the cell lysis protocol described in Example 1, the concentration of aP2 mRNA was determined. The results are presented in FIG. 3.

Also, the EC₅₀ results (the drug concentration at which the induced mRNA synthesis was inhibited by 50%) were found to be indistinguishable from parallel experiments using radioactive RNA dot-blot assay (data not shown). This Example demonstrates how easily researchers using the INVADER assay format of the present invention can determine the expression profile of cells treated with a large number of experimental variables.

### EXAMPLE 3

### Assaying NOS2 mRNA Expression Levels in Cells Exposed to Various Compounds

This example describes assaying NOS2 mRNA expression levels in cells exposed to various compounds using an INVADER assay. In particular, the INVADER assay may be employed to quantitate mRNAs directly from the cell lysate of human cancer, macrophage, neutrophil, hepatocyles, smooth muscel and endothelial cells.

Inducible nitric oxide synthase (iNOS or NOS2) is expressed in several cell types including macrophages, endothelial cells, and hepatocytes. In most cells, NOS2 is expressed only after induction by different stimuli such as cytokines. After induction the translated NOS2 enzyme produces high amounts of nitric oxide (NO). Macrophages use the high production of NO to kill bacteria, fungi, virus, parasites and tumor cells. Besides this important immune system function, NO seems to protect against liver injury and is important for skin wound healing. Overexpression of NO is causally involved in diabetes type I, rheumatoid arthritis, multiple sclerosis, inflammatory bowel disease, asthma, and septic shock.

In order to determine the effect of nutraceutical compounds on the production of NOS2 (iNOS) mRNA in various cells type, the various cell types may be assayed with the INVADER assay before being contacted with a nutraceutical. This establishes a baseline mRNA production level. An internal standard mRNA is also assayed, such as glyceraldehyde-3-phosphate dehydrogenase (GAPDH). Cells are then contacted with varying concentrations of various nutraceutical compounds, and then re-assayed with the INVADER assay to determine what the new level of mRNA production are (for both NOS2 and the internal standard). The baseline and new level are compared to determine what type of affect a particular nutraceutical has on a particular type of cells (the amount of mRNA can also be normalized for the different populations of cells by examining the mRNA levels of the internal standard). This information may then be employed to substantiate structure/functions claims for FDA purposes (e.g. under the Dietary Supplement Health and Education Act). In this regard, nutraceuticals that in fact have an impact on human cells may be identified and approved for sale.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method for testing a nutraceutical, comprising;
a) providing;
i) cells, wherein said cells express a baseline level of test gene mRNA,
ii) a nutraceutical; and
iii) INVADER assay detection reagents configured for detecting and quantitating said test gene mRNA; and
b) exposing said cells to said nutraceutical,
c) lysing said cells such that a cell lysate is generated, and
d) contacting said cell lysate with said INVADER assay detection reagents under conditions such that an assayed level of said test gene mRNA is determined.

2. The method of claim 1, further comprising step e) comparing said baseline level of said test gene mRNA to said assayed level of said test gene mRNA.

3. The method of claim 2, wherein said comparing generates nutraceutical activity data for said nutraceutical.

4. The method of claim 3, further comprising, step e) employing said nutraceutical activity data to substantiate a structure/function claim as used by the Dietary Supplement Health and Enforcement Act of 1994.

5. The method of claim 1, wherein said test gene mRNA comprises nitric oxide synthase mRNA.

6. The method of claim 5, wherein said nitric oxide synthase mRNA is human.

7. The method of claim 1, wherein said INVADER assay detection reagents comprise a probe and an INVADER oligonucleotide.

8. The method of claim 1, wherein said nutraceutical is classified as a Dietary Supplement under the Dietary Supplement Health and Education Act (DSHEA) of 1994.

9. The method of claim 1, wherein said lysing comprises heating said cells to a temperature of approximately 80-90 degrees Celsius.

10. A method for testing a nutraceutical, comprising;
a) providing;
i) cells, wherein said cells express a baseline level of test gene mRNA, and wherein said cells express a first level of an internal reference gene mRNA,
ii) a nutraceutical;
iii) INVADER assay detection reagents configured for detecting and quantitating said test gene mRNA and said internal reference gene mRNA; and
b) exposing said cells to said nutraceutical, and
c) lysing said cells such that a cell lysate is generated, and
d) contacting said cell lysate with said INVADER assay detection reagents under conditions such that an assayed level of said test gene mRNA is determined, and such that a second level of said internal reference gene is determined.

11. The method of claim 10, further comprising step e) comparing said first level of said internal reference gene to said second level of said internal reference gene, and comparing said baseline level of said test gene mRNA to said assayed level of said test gene mRNA.

12. The method of claim 11, wherein said comparing generates nutraceutical activity data for said nutraceutical.

13. The method of claim 12, further comprising, step e) employing said nutraceutical activity data to substantiate a structure/function claim.

14. The method of claim 10, wherein said test gene mRNA comprises nitric oxide synthase mRNA.

15. The method of claim 14, wherein said nitric oxide synthase mRNA is human.

16. The method of claim 10, wherein said nutraceutical is classified as a Dietary Supplement under the Dietary Supplement Health and Education Act (DSHEA) of 1994.

17. The method of claim 10, wherein said INVADER assay detection reagents comprise a probe and an INVADER oligonucleotide.

18. The method of claim 10, wherein said lysing comprises heating said cells to a temperature of approximately 80-90 degrees Celsius.

19. The method of claim 10, wherein said exposing, said lysing, and said contacting are performed in an automated manner.

20. A method for testing a nutraceutical, comprising;
a) providing;
i) a population of cells expressing test gene mRNA,
ii) a nutraceutical;
iii) INVADER assay detection reagents configured for detecting and quantitating said test gene mRNA; and
b) lysing a first portion of said population of cells such that a first cell lysate is generated,
c) contacting said first cell lysate with said INVADER assay detection reagents under conditions such that a baseline level of said test gene mRNA is determined,
d) exposing a second portion of said population of cells to said nutraceutical,
e) lysing said second portion of said population of cells such that a second cell lysate is generated, and
f) contacting said second cell lysate with said INVADER assay detection reagents under conditions such that an assayed level of said test gene mRNA is determined.

21. The method of claim 20, further comprising step g) comparing said baseline level of said test gene mRNA to said assayed level of said test gene mRNA.

22. The method of claim 21, wherein said comparing generates nutraceutical activity data for said nutraceutical.

23. The method of claim 22, further comprising, step e) employing said nutraceutical activity data to substantiate a structure/function claim as used by the Dietary Supplement Health and Enforcement Act of 1994.

24. The method of claim 20, wherein said test gene mRNA comprises nitric oxide synthase mRNA.

25. The method of claim 24, wherein said nitric oxide synthase mRNA is human.

26. The method of claim 20, wherein said nutraceutical is classified as a Dietary Supplement under the Dietary Supplement Health and Education Act (DSHEA) of 1994.

27. The method of claim 20, wherein said INVADER assay detection reagents comprise a probe and an INVADER oligonucleotide.

28. The method of claim 20, wherein said lysing comprises heating said cells to a temperature of approximately 80-90 degrees Celsius.

29. The method of claim 20, wherein said exposing, said lysing, and said contacting are performed in an automated manner.

30. A method for testing a nutraceutical, comprising;
a) providing;
i) a surface comprising a plurality of spatially discrete regions, wherein said spatially discrete regions comprise cells, wherein said cells express a baseline level of test gene mRNA
ii) at least one type of nutraceutical; and
iii) INVADER assay detection reagents configured for detecting and quantitating said test gene mRNA; and
b) adding said at least one type of nutraceutical to at least two of said plurality of spatially discrete regions,
c) lysing said cells in said at least two of said plurality of spatially discrete regions, and
d) contacting said at least two of said plurality of spatially discrete regions with said INVADER assay detection reagents under conditions such that an assayed level of said test gene mRNA is determined for said cells in each of said at least two of said plurality of spatially discrete regions.

31. The method of claim 30, further comprising step e) comparing said baseline level of said test gene mRNA to said assayed level of said test gene mRNA.

32. The method of claim 30, wherein said INVADER assay detection reagents comprise a probe and an INVADER oligonucleotide.

33. The method of claim 30, wherein said lysing comprises heating said cells to a temperature of approximately 80-90 degrees Celsius.

34. The method of claim 30, wherein said contacting is performed in a high throughput manner.

35. The method of claim 30, wherein said exposing, said lysing, and said contacting are performed in an automated manner.

36. The method of claim 30, wherein said plurality of spatially discrete regions are wells.
